(19) United States
(12) Patent Application Publication
(10) Pub. No.: US 2018/0187157 A1
(43) Pub. Date: Jul. 5, 2018

(54) HUMAN SERUM FOR CELL CULTURE MEDIUM FOR CLINICAL GROWTH OF HUMAN ADIPOSE STROMAL CELLS

(71) Applicant: Micheal MOELLER, Bensalem, PA (US)

(72) Inventor: Micheal MOELLER, Bensalem, PA (US)

(21) Appl. No.: 15/539,828

(22) PCT Filed: Dec. 31, 2015
(86) PCT No.: PCT/US15/68350

§ 371 Date: Jun. 26, 2017

(51) Int. Cl. C12N 5/077; C12N 5/0775

(52) U.S. Cl. CPC C12N 5/0653; C12N 5/0667; C12N 2501/135; C12N 2501/115; C12N 2501/11

(57) ABSTRACT

This invention details a cell culture medium for clinical growth of human adipose stromal cells for human clinical and therapeutic applications.

### CROSS REFERENCE TO RELATED APPLICATIONS

This application claims priority to U.S. Provisional Application No. 62/098,799 filed Dec. 31, 2014, incorporated herein in its entirety.

### FIELD OF THE INVENTION

The invention is directed to a cell culture medium for clinical growth of human adipose stromal cells for human clinical and therapeutic applications.

### BACKGROUND OF THE INVENTION

Adipose-derived stem cells, and, indeed, all mesenchymal stem cells are speculated to be perivascular, meaning that they grow in close proximity to blood vessels throughout the human body. This might indicate that ADSCs and MSCs are accustomed to a “bloody”, serum-rich environment. It is speculated that the addition of human serum will create a more natural medium for these cells. Substituting whole human serum for HSA will also greatly reduce medium cost.

There still exists today the need for a commercially viable stem cell culture medium for growth of human adipose stromal cells based on Human serum.

### BRIEF SUMMARY OF THE INVENTION

In a first embodiment, the invention is directed to a cell culture medium for clinical growth of human adipose stromal cells for human clinical and therapeutic applications, the medium including a basal medium suitable for mammalian cell culture; Human serum collected without anticoagulants and allowed to clot prior to serum processing; and

at least one of (i) growth promoting amounts of human insulin, (ii) human transferrin, (iii) human recombinant epidermal growth factor, (iv) human recombinant platelet-derived growth factor-BB, (v) basic fibroblast growth factor (FGF2) and (vii) phenol red; wherein the medium contains no Linoleic acid.

In another embodiment, the invention is directed to a method to make cell culture medium for clinical growth of human adipose stromal cells for human clinical and therapeutic applications including the steps of combing the components of a basal medium suitable for mammalian cell culture; human serum collected without anticoagulants and is allowed to clot prior to serum processing; and at least one of (i) growth promoting amounts of human insulin, (ii) human transferrin, (iii) human recombinant epidermal growth factor, (iv) human recombinant platelet-derived growth factor-BB, (v) basic fibroblast growth factor (FGF2) and (vii) phenol red; wherein the medium contains no Linoleic acid.

### DETAILED DESCRIPTION OF THE INVENTION

Certain terminology is used herein for convenience only and is not to be taken as a limitation on the present invention. The terminology includes the words specifically mentioned, derivatives thereof and words of similar import. The embodiments discussed herein are not intended to be exhaustive or to limit the invention to the precise form disclosed. These embodiments are chosen and described to best explain the principle of the invention and its application and practical use and to enable others skilled in the art to best utilize the invention.

Adipose-derived stem cells and all mesenchymal stem cells are speculated to be perivascular, meaning that they grow in close proximity to blood vessels throughout the human body. This indicates that ADSCs and MSCs are accustomed to a “bloody”, serum-rich environment. The present invention provides a cell growth medium with the addition of human serum which will create a more natural medium for cell growth and will also greatly reduce medium cost.

Normally, blood is collected in the presence of anticoagulants such as EDTA or sodium citrate. “Normal” serum created from this blood would, therefore, be expected to have all the normal clotting factors and structural proteins associated with blood clotting. This means that cells grown in normal serum could, conceivably, end up coated with clotting factors which could lead to blood clots following transplantation. “Off the clot” serum is collected without anticoagulants and is allowed to clot prior to serum processing; thus the clotting factors would not be therein.

The basic idea of using human serum capitalizes on the following principles: (i) MSCs (including ADSCs) are perivascular, meaning that they grow in close proximity to blood vessels throughout the human body. In fact, where capillaries are located, there are the endothelial cells which line the capillaries and the MSCs are immediately outside of the endothelial cells. This means that, throughout life, MSCs are going to be in the presence of serum and all that is in serum. One skilled in the art appreciates the idea of “defined medium” e.g. everything is added in known proportions and quantities. Further, one skilled in the art would recognize the natural environment is optimal for growing and sustaining these cells.

The present invention with the addition of some extra growth factors such as PDGF, EGF, and FGF2 is to create “supercharged” or “augmented” human serum, and this is what promotes the growth of MSCs in the medium. Serum contains large amounts of various substances, many of which are not completely characterized, and it is the unique blend of the component which results in the medium of the present invention. Human serum albumin could be used in the present invention, however, HAS is a large protein in serum to which many things bind such as growth factors, specific lipids, cytokines, etc. HSA is a carrier protein, and the lot-to-lot variability you see in HSA is likely due to differences in what's bound to the HSA. HSA is, unfortunately, expensive and shows a lot of “lot-to-lot” variability. Human serum is easier, and will include natural beneficial components that won't be in HSA.

Allogeneic serum from a healthy individual, may be able to exert a health-promoting effect on MSCs from someone not in great health. Conceivably, a patient-specific medium could be created by using autologous serum during the expansion process, keeping the process as autologous. Thus, basal medium could be sold with the user options of using either allogeneic or autologous serum depending on application.

As provided herein, in a first embodiment, the invention is directed to a cell culture medium for clinical growth of human adipose stromal cells for human clinical and therapeutic applications, said medium including a basal medium suitable for mammalian cell culture; human serum collected without anticoagulants and is allowed to clot prior to serum processing; and at least one of (i) growth promoting amounts of human insulin, (ii) human transferrin, (iii) human recombinant epidermal growth factor, (iv) human recombinant platelet-derived growth factor-BB, (v) basic fibroblast growth factor (FGF2) and (vii) phenol red; wherein the medium contains no Linoleic acid. The phenol red in the present invention is lower than in previous iterations of medium. One skilled in the art would appreciate phenol red is an estrogen mimic and it is desirable that it be used in reduced amounts. The population doublings provided herein were obtained in a low O2 environment (5% as opposed to the 20%+ of normal, ambient air).

The media is able to exceed 40 and even 50 doublings.

The media can achieve and sustain growth rates of 1 doubling/18-20 hours.

The media has multipotency sustained to at least 35-37.

The media can sustain growth for over 2 months before spontaneous differentiation occurs.

The media of the present invention is approximately 60% cheaper to make the media existing in the art. The ability to have cost efficient media product is core to the commercial success of the media, and therefore, the ability to build on the concepts of the present media to obtain improved media embodiment.

The main components of the base medium (DMEM:MCDB131:MCDB201, and the glutamine) may be pre-prepared into one basal medium the lab refers to a DMM. The components are obtained in their ‘raw’ form (ie. lyophilized) and reconstitute to the stock concentration listed.

The chart below lists the stock concentrations, not final concentrations listing of components to prepare the xeno-free adipose derived stem cell culture medium made with OTC serum includes:

| | Stock | | | mL per |
|---|---|---|---|---|
| | concentration/specification | Manufacture | Mft Ref # | Liter |
| Base Medium | | | | |
| Dulbecco's | low glucose, pyruvate, no | Life | 11054 | 555.6 |
| Modified Eagle | glutamine, no phenol red | technologies- | | |
| Medium | | GIBCO | | |
| MCDB 131 | Low glucose No glutamine | Life | 10372 | 185.2 |
| Medium | | technologies- | | |
| | | GIBCO | | |
| MCDB 201 | Comes as powder - prep'd by ACS | Sigma-Aldrich | M6770- | 185.2 |
| Medium | | | 10x1L | |
| GlutaMax ™ | 100x same as 200 mM | Life | 35050 | Directly |
| Supplement | L-Glutamine [but stable] (used 1 mL | technologies- | | added to |
| [Animal Origin- | per 100 mL of base media) | GIBCO | | each bottle |
| Free] | | | | of base media |
| Reagents & | | | | |
| Growth Factors | | | | |
| OTC Serum [xeno- | Off the clot Human AB Male | Access | Male | 50 |
| free] | Access Male Human AB Serum | Biologicals | Human | |
| | | | AB OTC | |
| | | | Serum | |
| ITSE | 100x Recombinant human Insulin | InVitria | 777ITS032 | 10 |
| [Animal Free] | 1.0 g/L, Recombinant human | | | |
| | transferrin 0.55 g/L, Sodium | | | |
| | selenite 0.00067 g/L, | | | |
| | ethanolamine 0.2 g/L | | | |
| L-ascorbate-2- | 10 mM - prep'd by ACS | Sigma-Aldrich | A8960-5G | 10 |
| phosphate | | | | |
| 2-Mercaptoethanol | 55 mM in Dulbecco's Phosphate | Life | 21985023 | 1.8 |
| | Buffered Saline | technologies- | | |
| | | GIBCO | | |
| recombinant | 10 μg/mL - prepared | PeproTech | AF-100-15 | 1 |
| human Epidermal | | | | |
| Growth Factor | | | | |
| [Animal Free] | | | | |
| recombinant | 10 μg/mL rhFGF-2 - prepared | PeproTech | AF-100-18B | 1 |
| human Fibroblast | | | | |
| Growth Factor- | | | | |
| basic [Animal | | | | |
| Free] | | | | |
| recombinant | 5 μg/mL - prepared | PeproTech | AF-100-14B | 1 |
| human Platelet- | | | | |
| Derived Growth | | | | |
| Factor-BB | | | | |
| Dexamethasone | 10 μM - prepared | Sigma-Aldrich | D9184-100MG | 0.1 |

To ensure sterility all components are added in the order listed to the top half of a 1000 mL filter unit with reservoir (filter is 0.22 μm pore size and low binding (PES)). Completed media has a shelf life of 3 weeks and is stored in refrigerator (4-8° C.)

Storage of components is preferred in the following manner; Base Media, GlutaMax, ITSE, L-ascorbate-2-phosphate, 2-Mercaptoethanol, Dexamethasone—refrigerator (4-8° C.). The OTC Serum is in a freezer −20° C. or colder, aliquots are stored in the ultra-low freezer (−80±10° C.). Prepared Growth Factors; EGF, FGF-2, PDGF-BB are stored in the ultra-low Freezer (−80±10° C.).

One skilled in the art would recognize the importance of the collection, processing and storage of cells prior to expansion in the medium of the present invention. Most particularly, the ability to collect and digest cells (and storage if necessary) in a method which preserves the characters and properties of the cells, e.g. markers, is required to ensure the interaction of the components of the media of the present invention with the cells for expansion. The cells expanded in the medium of the present invention were processed from adipose tissue by the methods found in U.S. Ser. No. 13/646,647, incorporated herein in its entirety (American Cryostem Corporation, Eatontown, N.J.). The resultant cells processed from adipose tissue by methods in the '647 application provide a unique set of defined markers, properties and characteristics which ensure the synergistic effect and resultant properties, as defined herein, upon expansion in the medium of the present invention.

### EXAMPLES

The example was prepared as described by methods for preparation of cell culture recognized by those skilled in the art.

### Example 1

| 92.51 ml DMM | 92.51% | |
|---|---|---|
| 5.00 ml Off-The-Clot Human Serum | 5.00% | |
| 1.00 ml ITSE | 1.00% | |
| 1.00 ml Ascorbate-2-Phosphate Solution | 100 | μM |
| 0.18 ml 2-Mercaptoethanol | 100 | μM |
| 0.10 ml EGF | 10 | ng/ml |
| 0.10 ml FGF2 | 10 | ng/ml |
| 0.10 ml PDGF-BB | 5 | ng/ml |
| 0.01 ml Dexamethasone Solution | 1 | Nm |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A cell culture medium for clinical growth of human adipose stromal cells for human clinical and therapeutic applications, said medium comprising:
a basal medium suitable for mammalian cell culture;
human serum collected without anticoagulants and allowed to clot prior to serum processing; and
at least one of (i) growth promoting amounts of human insulin, (ii) human transferrin, (iii) human recombinant epidermal growth factor, (iv) human recombinant platelet-derived growth factor-BB, (v) basic fibroblast growth factor (FGF2) and (vii) phenol red; wherein the medium contains no Linoleic acid.

2. The media of claim 1, wherein the media is able to exceed 40 and even 50 doublings.

3. The media of claim 1, wherein to achieve and sustain growth rates of 1 doubling/18-20 hours.

4. The media of claim 1, multipotency sustained to at least 35-37.

5. The media of claim 1, sustain growth for over 2 months before differentiation occurs.

6. A method to make cell culture medium for clinical growth of human adipose stromal cells for human clinical and therapeutic applications consisting of the steps of combing the components of a basal medium suitable for mammalian cell culture; human serum collected without anticoagulants and is allowed to clot prior to serum processing; and
at least one of (i) growth promoting amounts of human insulin, (ii) human transferrin, (iii) human recombinant epidermal growth factor, (iv) human recombinant platelet-derived growth factor-BB, (v) basic fibroblast growth factor (FGF2) and (vii) phenol red.

7. A process to extend the growth and existence of multipotency of mesenchymal stem cells comprising the steps of:
a. preparing a growth medium of claim 1;
b. expanding the mesenchymal stem cells in the medium of a.

8. Mesenchymal stem cells made by the process of claim 7, wherein the cells are capable of expansion to 40+ doublings, and growth can be extended for up to 2 months before spontaneous differentiation occurs.

9. Mesenchymal stem cells made by the process of claim 7, wherein multipotency exists at 35 to 50 doublings.

10. Mesenchymal stem cells made by the process of claim 7, wherein mesenchymal stem cells are adipose derived stem cells wherein the undifferentiated cells have the following phenotype: CD14−, CD19−, CD29+, CD31−, CD34−, CD44+, CD45−, CD49d+, CD73+, CD90+, CD105+, and CD146+.

11. Mesenchymal stem cells made by the process of claim 7, wherein the cells differentiate readily into Oil Red+ adipocytes, Alcian Blue+ chondrocytes, and Alizarin Red+ osteocytes.
